(19) United States
(12) Patent Application Publication
(10) Pub. No.: US 2012/0184904 A1
(43) Pub. Date: Jul. 19, 2012

(54) ELECTRO MECHANICAL PRESSER WITH RIBBED BOTTLE FOR INFUSION OF LIQUIDS

(76) Inventor: Fernando Cesar Toniazzi Lissa, Criciuma (BR)

(21) Appl. No.: 13/087,875

(22) Filed: Apr. 15, 2011

(30) Foreign Application Priority Data
Apr. 15, 2010 (BR) PI 10011156-0

(51) Int. Cl. A61M 5/142

(52) U.S. Cl. 604/131

(57) ABSTRACT

The present invention relates to an ELECTRO MECHANICAL PRESSER WITH RIBBED BOTTLE FOR INFUSION OF LIQUIDS. The invention comprises a presser holder through which a mechanism connected to an electric motor presses a ribbed bottle containing liquid to be administered to a patient, thereby providing a continuous infusion of the same. The invention solves the problem of disruption of liquid infusion attributable to the creation of a vacuum in the bottle.

### CROSS-REFERENCE TO RELATED APPLICATION

This application claims the benefit of the priority filing date of Brazilian patent application no. PI 1001156-0 filed on Apr. 15, 2010.

### FEDERALLY SPONSORED RESEARCH

Not Applicable

### SEQUENCE LISTING OR PROGRAM

Not Applicable

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

1. Field of the Invention

The present invention pertains, in general, to the technology sector of hospital equipment and refers, more specifically, to a new concept applied to equipment for facilitating the infusion of fluids in a patient's body.

2. Background of the Invention

The state of art of this technology sector includes bottles for infusion of fluids in a patient's body, but these conventional devices lack efficiency because they do not allow a continuous infusion. This is due mainly to the shape of the bottle that creates an internal vacuum and lateral compression when the bottle is emptied. An example of these devices is Brazilian patent application number BR MU8301768 filled on Apr. 4, 1983.

3. Novelty of the Invention

The invention disclosed in this specification aims to achieve a continuous infusion with no disruption attributable to the creation of a vacuum in the bottle, using high capacity saline infusion equipment.

The above objective is achieved in the form of a new equipment design for fluid infusion that employs an electromechanical presser associated with a ribbed bottle capable of providing a continuous infusion and high capacity.

### SUMMARY

The present invention relates to an Electro Mechanical Presser with Ribbed Bottle for infusion of liquids in a patient's body, comprising a presser holder through which a mechanism connected to an electric motor presses a ribbed bottle containing liquid, thereby providing a continuous infusion of the same. The invention solves the problem of disruption of liquid infusion attributable to the creation of a vacuum in the bottle.

### DRAWINGS

FIG. 1 perspective view of the whole invention.

FIG. 2 elevational side view of the presser support.

FIG. 3 elevational front view of the presser support.

FIG. 4 elevational front view of the ribbed bottle for liquids.

FIG. 5 bottom view of the ribbed bottle for liquids.

FIG. 6 elevational side view of the assembled invention.

FIG. 7 elevational front view of the invention without the ribbed bottle.

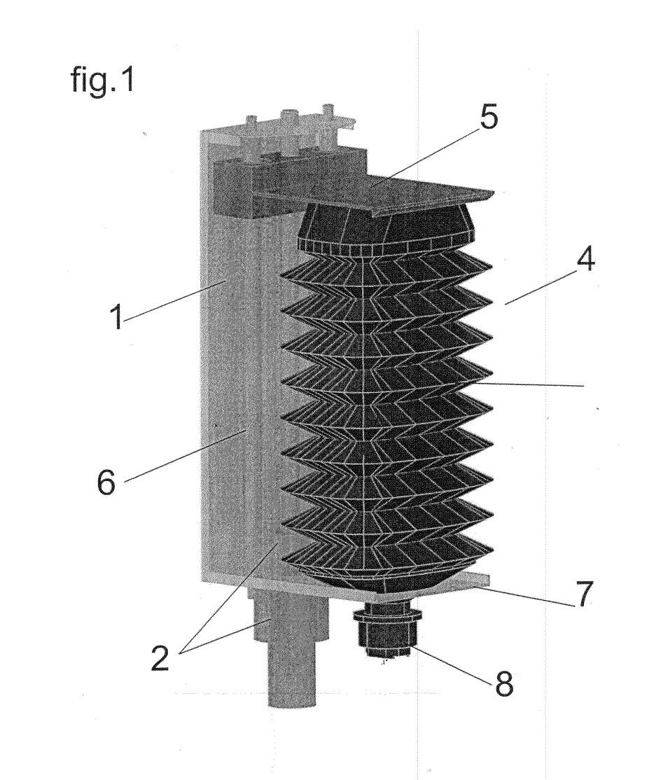

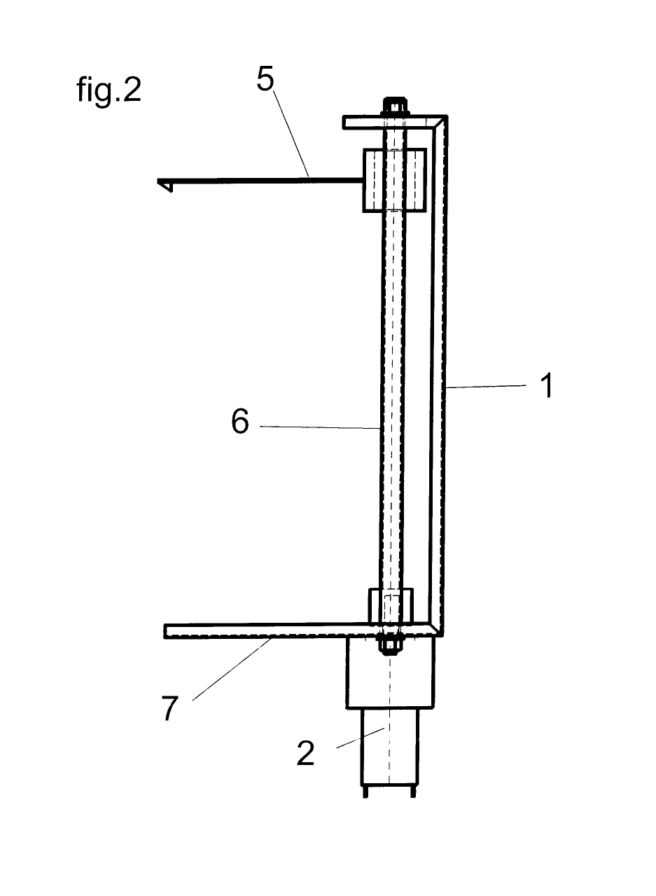

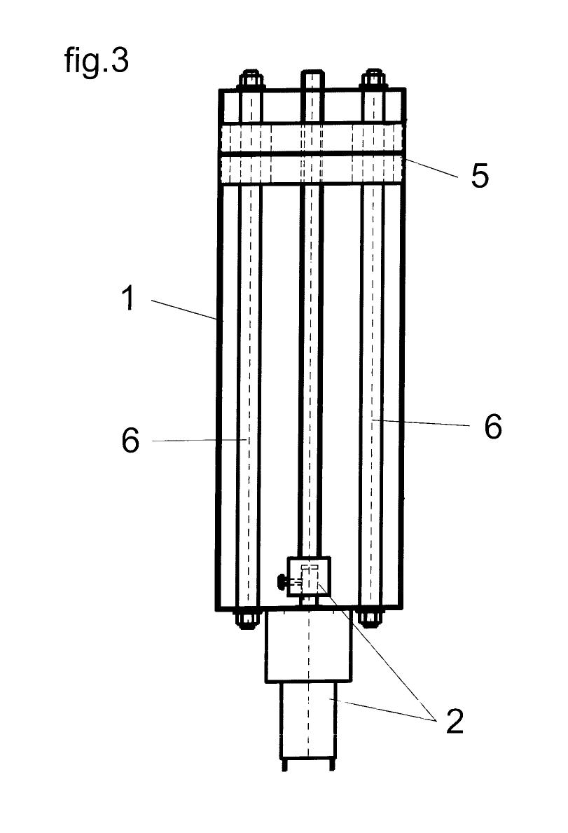

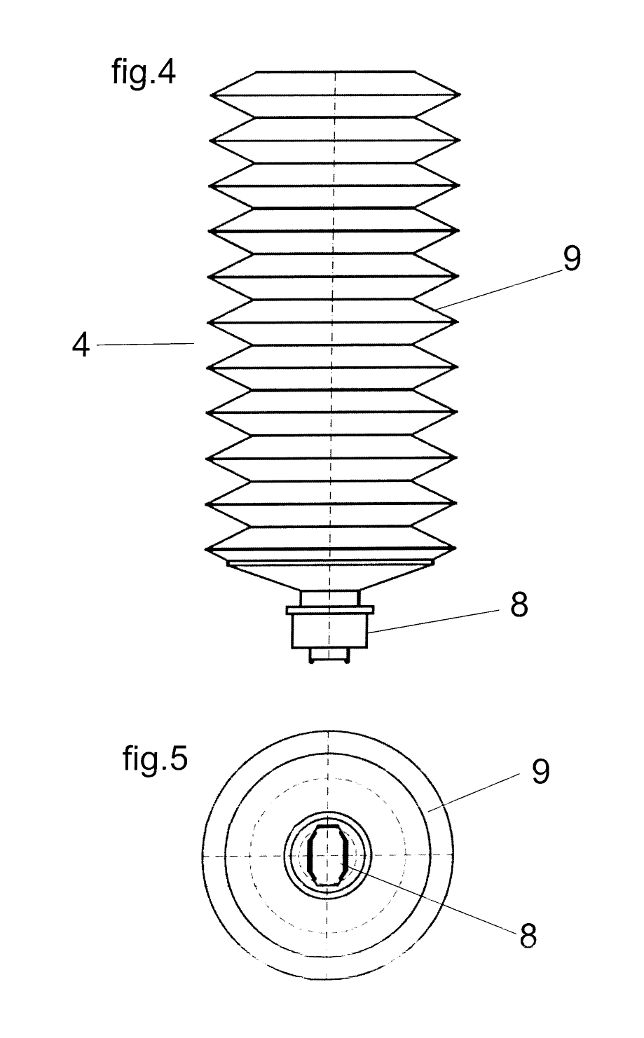

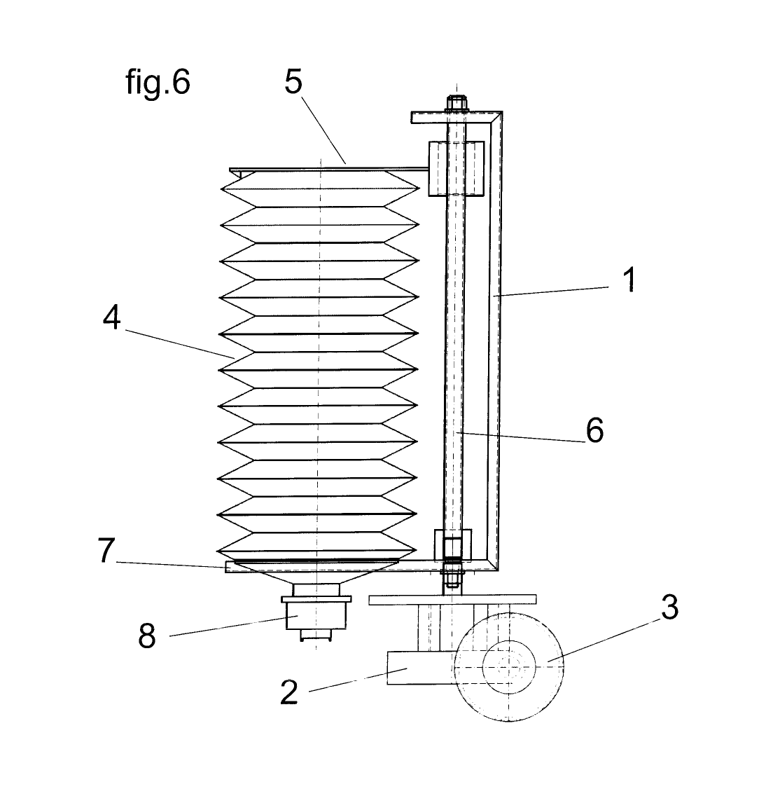

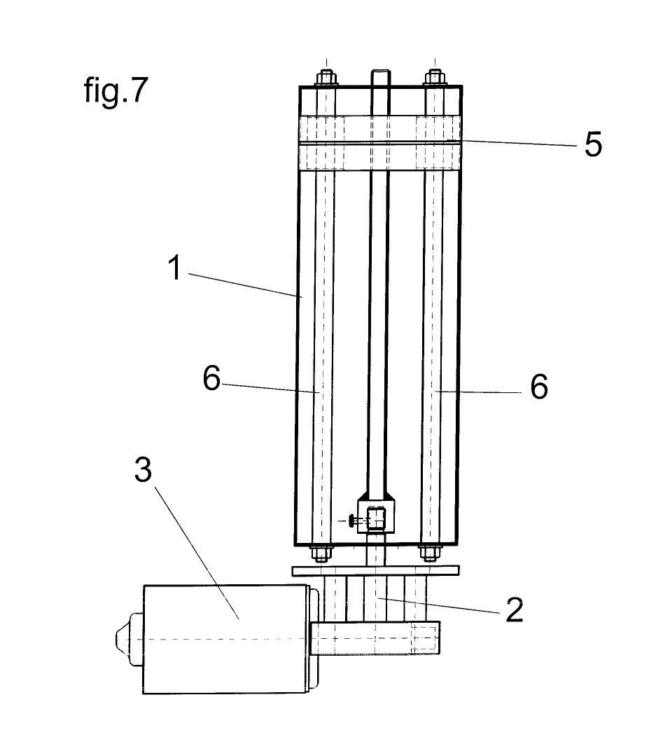

### DETAILED DESCRIPTION

The present invention is an electro mechanical presser with a ribbed bottle for infusion of liquids comprising a presser holder (1), which through a mechanism (2) connected to an electric motor (3), presses a ribbed bottle (4) containing liquid to be administered to a patient, providing a continuous infusion of said liquid.

In the preferred embodiment of the invention, the presser holder (1) comprises a movable plate (5), driven by an electric motor (3), which defines a vertical upward and downward path supported by guides (6) and compresses the bottle (4), and also comprising a base or cradle (7) to receive an outlet nozzle (8) of said bottle (4).

The bottle (4) is made of a flexible and resilient material and has a ribbed body (9), which, when compressed, continuously dispenses the liquid, preventing lateral deformation and formation of a vacuum through the outlet nozzle (8).

It is understood that all aspects of the invention are not limited to the specific depictions, configurations or relative proportions set forth herein which depend upon a variety of conditions and variables. Various modifications in form and detail of the embodiments of the invention are apparent to a person skilled in the art. It is therefore contemplated that the invention shall also cover any such modifications, variations and equivalents.

What is claimed is:

1. An ELECTRO MECHANICAL PRESSER WITH RIBBED BOTTLE FOR INFUSION OF LIQUIDS comprising a presser holder (1), through which a mechanism (2) connected to an electric motor (3) presses a ribbed bottle (4) containing liquid to be administered to a patient, providing a continuous infusion of the same.

2. The ELECTRO MECHANICAL PRESSER WITH RIBBED BOTTLE FOR INFUSION OF LIQUIDS of claim 1, wherein the presser holder (1) comprises an electric engine-driven (3) movable plate (5) that defines a vertical upward and downward path to compress the bottle (4).

3. The ELECTRO MECHANICAL PRESSER WITH RIBBED BOTTLE FOR INFUSION OF LIQUIDS of claim 2, wherein the movable plate is associated with guides (6).

4. The ELECTRO MECHANICAL PRESSER WITH RIBBED BOTTLE FOR INFUSION OF LIQUIDS of claim 2, wherein the presser holder (1) also comprises a base or cradle (7) to receive an outlet nozzle (8) of said bottle (4).

5. The ELECTRO MECHANICAL PRESSER WITH RIBBED BOTTLE FOR INFUSION OF LIQUIDS of claim 1, wherein the bottle (4) is made of a flexible and resilient material and has a ribbed body (9).
